(19) United States
(12) Patent Application Publication
(10) Pub. No.: US 2015/0166255 A1
(43) Pub. Date: Jun. 18, 2015

(54) VITAMIN B-12 INJECTION KIT

(71) Applicant: ASCLEMED USA, INC. dba EnovaChem Manufacturing, Torrance, CA (US)

(72) Inventor: Robert Prentiss Nickell, Torrance, CA (US)

(73) Assignee: ASCLEMED USA, INC. dba EnovaChem Manufacturing, Torrance, CA (US)

(21) Appl. No.: 14/562,583

(22) Filed: Dec. 5, 2014

Related U.S. Application Data
(60) Provisional application No. 61/914,958, filed on Dec. 12, 2013.

(51) Int. Cl. B65D 85/00; A61F 13/00; A41D 13/11; A41D 19/00; B65D 5/64; B65D 1/09

(52) U.S. Cl. CPC B65D 85/70; B65D 5/64; B65D 1/09; A41D 13/11; A41D 19/0055; A61F 13/00

(57) ABSTRACT

Provided is a kit comprising a compound having vitamin B-12 activity in a form that is suitable for injection. The kit can be in the form of a box with a lid with a number of components placed in the box. The compound having vitamin B-12 activity can be cyanocobalamin, typically in form of a solution in a vial. The kit can contain an alcohol pad. The kit can contain a pair of gloves. The kit can contain a csr wrap. The concentration of the cyanocobalamin can be 1000 mcg/ml. In one embodiment, the kit is a box with a lid and following components placed inside of the box: a) a container having 1 milliliter (mL) of a solution of cyanocobalamin with a concentration of 1000 mcg/ml; b) isopropyl pad; c) a pair of gloves and d) a csr wrap.

### CROSS-REFERENCE

The present application claims the benefit of provisional patent application No. 61/914,958 tiled on Dec. 12, 2013, which is incorporated herein by reference in its entirety.

### BACKGROUND SECTION OF THE INVENTION

With the changing reimbursements, types of health care insurance, and new regulations with regards to treatment, a medical practitioner and his/her team when treating a patient is under immense time pressures to perform quickly, efficiently and with safety for the patient. If a patient requires a singular or even multiple injections, the medical practitioner must rely on his/her team to first locate the necessary and correct injectable medications, insure they are the correct strength and percentage, insure dating is current, insure the top has not been contaminated, insure the liquid is not crystallized, or containing growth, insure there is sufficient medication in the vial, and insure that the injection has been stored at the proper temperature and humidity as well as all other possible related materials and safety precautions needed to treat the patient. The medical practitioner can lose valuable time trying to not only locate the necessary drugs and materials to treat the patient but as well to insure the compliance, the safety of the patient by preventing contamination. In some instances, not all the drugs and materials may be readily available, in the current exam room, resulting in delayed treatment and possible and potential medical errors. In addition, the medical practitioner or his/her team may make a mistake by incorrectly choosing the drug, by incorrectly identifying the product, not checking the expiration date, not returning it to the proper environment for storage, or not having enough product and thus requiring an additional sterile vial to be opened to inject directly into the veins of a patient.

There is a need in the art for a convenience kit that solves the above mentioned problems in the medical practitioner office.

### SUMMARY SECTION OF THE INVENTION

Provided is a kit comprising a first container and following components placed inside of the first container; a) a second container of a compound having vitamin B-12 activity in a form that is suitable for injection; b) an alcohol pad; c) a pair of gloves; and d) csr wrap. The compound having vitamin B-12 activity can be cyanocobalamin. The cyanocobalamin can have a concentration of 1000 mcg/ml. A 1 ml solution of the cyanocobalamin can be present in the second container. The first container can be a box. The first container can be a box with a lid. The alcohol pad can be an isopropyl pad. The kit can further comprise a face mask. The kit can further comprise a bandage. The second container can be a vial. The kit can have an instruction sheet.

Provided is a kit comprising a box with a lid and following components placed inside of the box: a) a vial holding 1 milliliter (mL) of a solution of cyanocobalamin with a concentration 1000 mcg/ml; b) isopropyl pad; c) a pair of gloves; and d) a csr wrap.

### BRIEF DESCRIPTION OF THE FIGURES

FIG. 1 illustrates a box where a plurality of components are placed therein.

FIG. 2 illustrates a box in which an ampoule or a vial can be placed.

FIG. 3 illustrates a vial.

FIG. 4 illustrates an ampoule.

FIG. 5 illustrates a glove.

FIG. 6 illustrates an alcohol pad.

FIG. 7 illustrates a package and a strip bandage.

FIG. 8 illustrates a face mask.

FIG. 9 illustrates an instruction sheet.

FIG. 10 illustrates a CSR wrap.

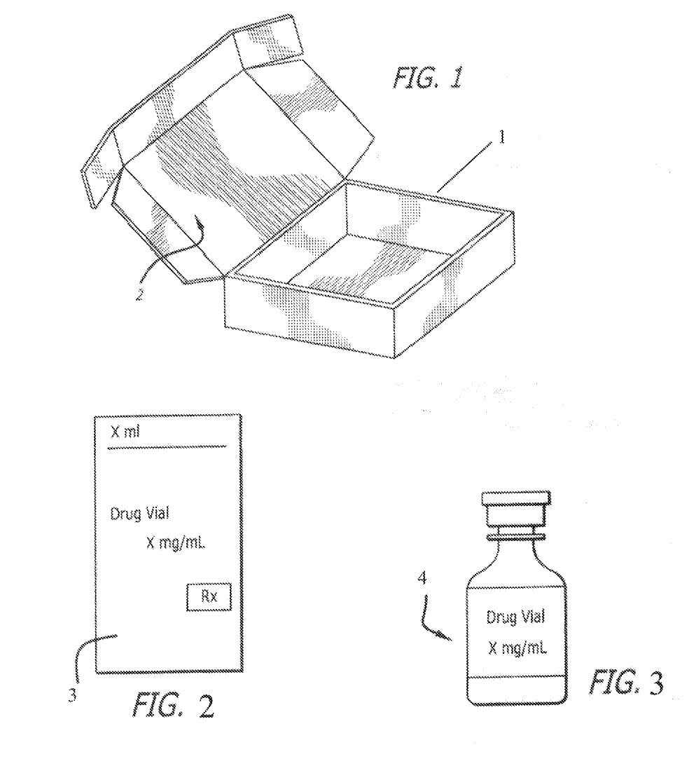

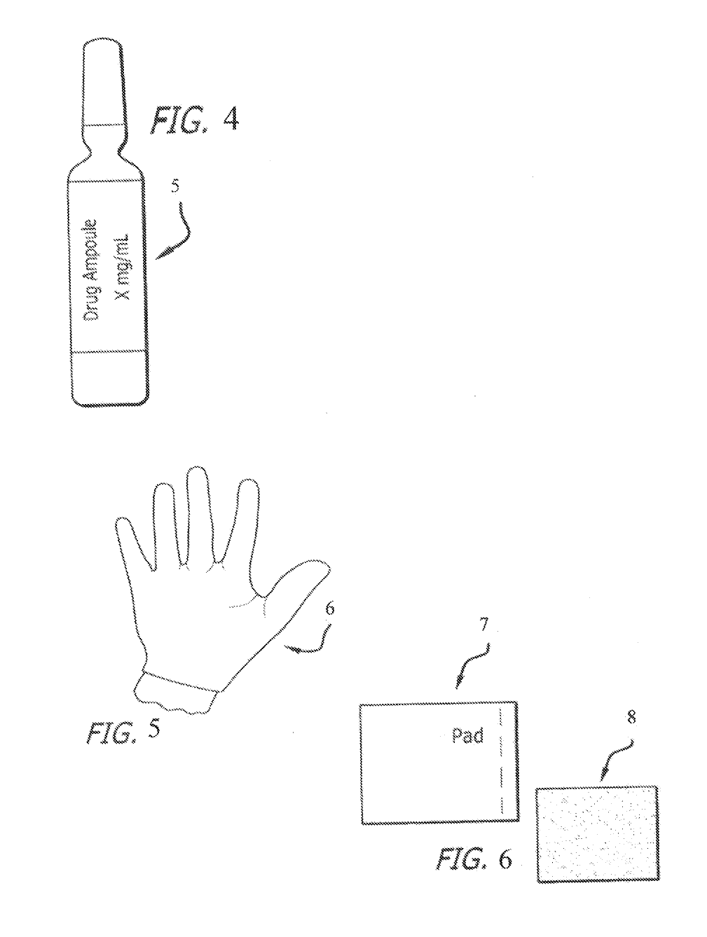

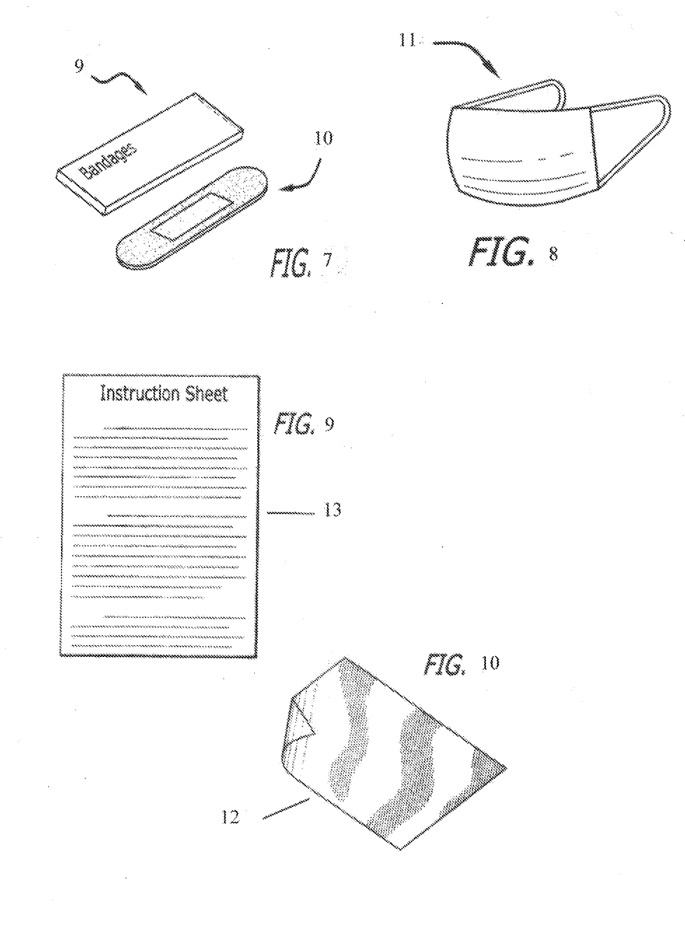

### DETAILED DESCRIPTION OF THE INVENTION

The present invention provides a kit for injection of vitamin B-12 to a patient. The kit allows a medical practitioner to accurately and quickly perform his or her function, while reducing medical errors. The kit can be used for treatment of pernicious anemia and vitamin B12 deficiency. It also can be used in the Schilling test to determine vitamin B12 absorption.

One component of the medical kit can be a compound with vitamin B-12 activity in a pharmaceutical dosage form that is suitable for injection. Cyanocobalamin (Cobolin-M®) (CAS No. 68-19-9) is the most common and widely produced form of the chemical compounds that have vitamin B-12 activity. Vitamin B12 is the generic descriptor name for any of such vitamers of vitamin B-12. The body can convert cyanocobalamin to any one of the active vitamin B-12 compounds. The dose of the cyanocobalamin can be about 1000 mcg/ml (microgram/ml). About 1 ml of this dose can be present in the kit.

Cyanocobalamin Injection, USP (US Pharmacopeia) (is a sterile solution of cyanocobalamin for intramuscular or subcutaneous injection. Each milliliter (mL) contains 1000 mcg cyanocobalamin. Each vial can also contain Sodium Chloride, 0.9%, Benzyl Alcohol, 1.5%, is present as a preservative. Sodium hydroxide and/or hydrochloric acid may have been added during manufacture to adjust the (range 4.5-7.0).

A container (ampoule 5 or vial 4) holding the cyanocobalamin in as form that is ready for injection is placed inside of the kit. The ampoule 5 or vial 4 can be placed in its own box 3.

One component of the medical kit can be alcohol pads 8, which is an antiseptic for preparation of the skin prior to injection. The alcohol pads 8 can be individually foil wrapped 7. The alcohol swabs can contain 70% isopropyl alcohol. The alcohol pad can come in its own foil wrap 7, which the medical practitioner would tear to remove the pad 8.

One component of the medical kit can be a pair of sterile gloves 6. The gloves 6 can be any suitable glove that is used by a medical practitioner, such as latex or nitrile gloves. In one embodiment, a pair of nitrite gloves are part of the kit. Nitrile gloves are a type of disposable glove made of Nitrile rubber, also known as Buna-N, Perbunan, or NBR. Nitrite gloves contain no latex. A medical practitioner can tear a package containing the gloves 6 and wear the gloves 6 before performing an injection to help prevent contamination and increase compliance.

One component of the kit can be a CSR (Central Supply Room) wrap 12. The CSR wrap 12 can be put on a tray or a surface before placement of the other components of the kit. The CSR wrap 12 keeps a clean environment. The CSR wrap 12 can be in its own wrapping or box.

The kit can contain an instruction sheet 13. The instruction sheet 13 can identify one or more components of the kit, provide instructions as to how to use each component, and optionally suggest an order of use for the components of the kit. The instruction sheet 13 can provide instructions as to where and how to inject each of the active ingredients.

Instead of an instruction sheet, each component of the kit can be described on the box 1 of the kit, for example on the sides of the box 1. Each component can have its own separate instruction sheet provided by its own manufacturer.

The components of the kit can be placed in a container, which is typically a box 1. The box 1 can be a rectangular cube which has a lid 2 on top for opening the box.

Other components of the kit can include a face mask 11 and bandage (Band-Aid®) 9, 10.

The following table lists the components of an exemplary kit:

Kit #1

Cyanocobalamin injection USP 1000 mcg/ml (1 ml) × 1
Isopropyl alcohol 70% prep pad × 1
Pair of sterile latex gloves-size 7 × 1
1 csr wrap

What is claimed is:

1. A kit comprising a first container and following components placed inside of the first container:
a) a second container of a compound having vitamin B-12 activity in a form that is suitable for injection;
b) an alcohol pad;
c) a pair of gloves; and
d) a csr wrap.

2. The kit of claim 1, wherein the compound having vitamin B-12 activity is cyanocobalamin.

3. The kit of claim 1, wherein the cyanocobalamin has a concentration of 1000 mcg/ml.

4. The kit of claim 3, wherein a 1 ml solution of the cyanocobalamin is present in the second container.

5. The kit of claim 1, wherein the first container is a box.

6. The kit of claim 5, wherein the first container is a box with a lid.

7. The kit of claim 1, wherein the alcohol pad is isopropyl pad.

8. The kit of claim 1, further comprising a face mask

9. The kit of claim 1, further comprising a bandage.

10. The kit of claim 1, wherein the second container is a vial.

11. The kit of claim 1, further comprising an instruction sheet.

12. A kit comprising a box with a lid and following components placed inside of the box:
a) a vial holding 1 milliliter (mL) of a solution of cyanocobalamin with a concentration of 1000 mcg/ml;
b) isopropyl pad;
c) pair of gloves; and
d) a csr wrap.
